(19) United States
(12) Patent Application Publication
(10) Pub. No.: US 2009/0171298 A1
(43) Pub. Date: Jul. 2, 2009

(54) SYRINGE WITH EXTENDABLE AND RETRACTABLE NEEDLE

(75) Inventor: Luc Magne, Victoria (CA)

Correspondence Address: KLARQUIST SPARKMAN, LLP, 121 SW SALMON STREET, SUITE 1600, PORTLAND, OR, 97204, US

(73) Assignee: University of Victoria Innovation and Development Corporation

(21) Appl. No.: 12/334,415

(22) Filed: Dec. 12, 2008

Related U.S. Application Data
(63) Continuation-in-part of application No. 12/002,117, filed on Dec. 13, 2007.

(51) Int. Cl. A61M 5/315

(52) U.S. Cl. 604/232; 604/218

(57) ABSTRACT

A syringe for use with a carpule and needle is provided. The syringe comprises: (i) a holder for accepting the carpule, the holder comprising at least two linearly disposed first mating members; (ii) a plunger for engagement with the holder; and (iii) a shell for slidably housing the holder, the shell having a distal aperture and a proximal aperture, with a bore therebetween, the shell comprising at least one integral biased member proximate to the proximal aperture, the biased member having a second mating member proximate to a distal end, for mating with the first mating member, such that in a first position, the needle is housed within the shell and in a second position, the needle extends at least partially from the shell.

This application is a Continuation-in-Part of co-pending U.S. application Ser. No. 12/002,117, filed Dec. 13, 2007, herein incorporated by reference.

### TECHNICAL FIELD

The present invention relates to a syringe for use with a carpule and needle, more specifically, to a retractable syringe that permits the needle to be obscured from view as desired.

### BACKGROUND ART

In recent years there has been growing concern related to the safety of syringes. Conventional needles are provided with a cap or cover which is removed once the needle is affixed to the syringe. At this time, there is a potential for the practitioner or the patient to be inadvertently stuck with the needle or for contamination of the needle. Syringes with retractable needles have been developed to overcome these issues and enable the practitioner to expose the needle only when necessary.

Typically, a retractable needle has two positions, retracted and extended. The needle must be placed in the fully extended position in order to prepare the needle for administration of the injection by depression of a plunger and dispensing of the carpule contents. In addition, many retractable needles such as the SMART SYRINGE® (SMART SYRINGE is a registered trademark of Sakharam Mahurker, Chicago, Ill., USA) provide only a single use and are not environmentally friendly.

In the field of dentistry, injections are a common fear identified in dental patients. Recent studies have shown that both the patient and the dentists may be traumatized on viewing the needle portion of a syringe when receiving dental syringe injections. There have many dental syringes developed specifically to reduce or eliminate patient anxiety on receiving injections. These syringes are typically marketed as “patient friendly” syringes, however “patient friendly” has often referred to a reduced time period associated with receiving an injection not needle related anxiety.

The Wand® (The Wand is a registered trademark of Milestone Scientific Inc., Livingston, N.J., USA) is a computer controlled dental injection system that is intended to alleviate or reduce patient pain and anxiety associated with receiving injections by controlling the speed at which the injection is administered by a computer. The Wand® resembles a ballpoint pen and is easier for dentists to handle because its light weight. However, this device is both costly to purchase and operate, and time consuming to prepare and perform the injection.

The Comfort Control® Syringe (Comfort Control is a registered trademark of Midwest Dental Products Corp., Des Plaines Ill., USA) was developed to deliver a more predictable and comfortable injection in sensitive areas such as the palate or front of the mouth. The syringe is wired to a microprocessor which directs a steady, slow injection regardless of tissue density.

Another device, the Vibraject provides a motor attached to a conventional syringe that causes the needle portion of the syringe to vibrate. The high vibration of the needle stimulates the nerve endings and blocks the transmission of pain to the brain.

### SUMMARY

Exemplary embodiments of the present invention relate to a syringe assembly having an extendable and retractable outer shell for exposing a needle.

An exemplary embodiment relates to a syringe assembly comprising a holder for receiving a container having a medicament, a plunger slidably received in the holder and an outer shell having an inner surface where a portion of the inner surface slidably engages an outer surface of the holder. A forward end of the plunger selectively engages the container to dispense the medicament through a needle. The outer shell is movable between an extended position and a retracted position for exposing the needle. The plunger and the holder are movable as a unit relative the outer shell when the outer shell moves between the extended position and the retracted position. When the outer shell is in the retracted position, the plunger is movable to a dispensing position.

In one embodiment, a proximal end of the outer shell is biased toward the holder. The proximal end may include a protrusion for selectively engaging a depression in the outer surface of the holder, and the outer shell is then biased toward the depression.

An alternative exemplary embodiment relates to an outer shell for a syringe comprising an inner surface, a portion of said inner surface slidably engaging an outer surface of a holder and a proximal end biased toward the holder for selectively engaging the outer surface of the holder. The holder is configured for receiving a container having a medicament. The outer shell is movable between an extended position and a retracted position for exposing a needle. When the outer shell moves between the extended position and the retracted position, a plunger slidably received in the holder, and the holder are movable as a unit relative to the outer shell. When the outer shell is in the retracted position, a forward end of the plunger is movable relative to the holder in a dispensing direction to dispense the medicament through the needle.

In one embodiment, the proximal end further includes a protrusion for selectively engaging a depression in the outer surface of the holder. The protrusion of the outer shell is biased toward the depression.

In another embodiment, the outer surface of the holder has a first depression and a second depression. When the protrusion engages the first depression, the position of the outer shell corresponds to the extended position. When the protrusion engages the second depression, the position of the outer shell corresponds to the retracted position. In a further embodiment, the protrusion is formed on an inner wall of the outer shell.

In another embodiment, the holder further includes a channel formed in the outer surface. The channel continuously engages the protrusion of the outer shell. In yet another embodiment, the depression is formed in the channel.

In one embodiment, the outer surface of the holder further includes at least one intermediate depression. In another embodiment, the outer surface of the holder further includes two intermediate depressions. In yet another embodiment, the outer surface of the holder further includes a plurality of intermediate depressions. When the protrusion engages an intermediate depression, the position of the outer shell corresponds to a partially retracted position.

In one embodiment, an outer wall of the outer shell has a window formed therein.

In another embodiment, the syringe assembly further includes a spring biased toward the container for resiliently retaining the container in the holder.

In one embodiment, the outer shell is transparent. In another embodiment, a portion of a distal end of the outer shell is textured to obscure the needle.

In one embodiment, the syringe assembly is self-aspirating.

In a further embodiment, the outer shell is constructed of a suitable flexible material that is temporarily deformable. The suitable flexible material is selected from the group consisting of plastics, metals, compliant polymeric material and combinations thereof. The compliant polymeric material may be shaped into a child-friendly shape, for example an animal head, an animal, or a tooth. A portion of the proximal end of the outer shell may further include an outer coating of a compliant polymeric material.

In one embodiment, the outer shell is injection molded. Alternatively, the syringe assembly may be injection molded.

The foregoing and other features and advantages of the syringe assembly will become more apparent from the following detailed description, which proceeds with reference to the accompanying figures.

### BRIEF DESCRIPTION OF THE DRAWINGS

Embodiments of the present invention will now be described, by way of example only, with reference to the attached Figures, wherein:

FIG. 1 is an isometric view of an embodiment of a syringe assembly with a needle extended;

FIG. 2 is an isometric view of the syringe assembly shown in FIG. 1 with the needle retracted;

FIG. 3 is an isometric view of a portion of the syringe assembly of FIG. 1 having an outer shell removed;

FIG. 4 is a portion of the syringe assembly shown in FIG. 1 in section showing depressions in a holder of the syringe;

FIG. 5 is a top view, partially in section, of an exemplary embodiment of the syringe assembly; and

FIG. 6 is a top view, partially in section, of an exemplary embodiment of the syringe assembly; and

FIG. 7 is an isometric view of a portion of the syringe assembly shown in FIG. 2 showing a section of an inner wall of the outer shell.

### DETAILED DESCRIPTION OF THE INVENTION

Some exemplary embodiments of the present invention relate to a syringe having a needle that is retractable and extendable such that the needle may be advanced through a series of specific needle positions, until the needle is fully extended.

A carpule syringe facilitates repeated use, specifically repeated loading of carpules and enables easy insertion of doses of a medicament and discharge of exhausted containers. A carpule is a container or cartridge having a medicament therein. A carpule syringe is characterized by the absence of a fixed barrel in which a piston reciprocates. Instead, a carpule syringe includes a shell with an opening designed to receive a medicament filled carpule, the ends of which are closed and hermetically sealed. The frame is provided with a reciprocatable shaft arranged to engage with one end of the carpule such that when the shaft is pushed forwardly in the frame, it will apply pressure to the liquid medicament within the carpule so as to dispense the same through a needle.

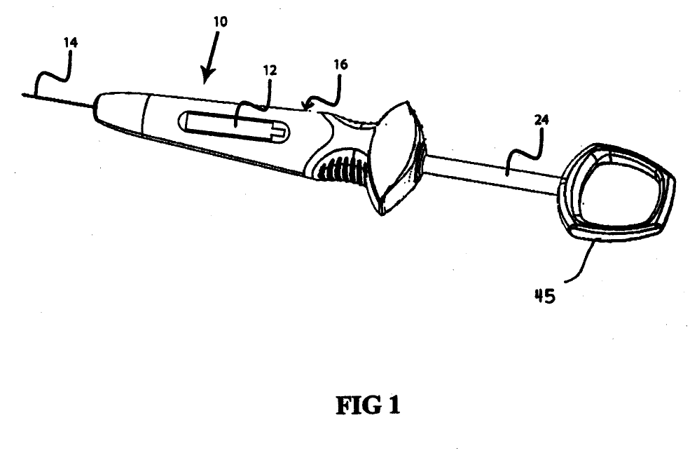

Referring to FIG. 1, an isometric view of an embodiment of a syringe assembly 10 with a needle 14 extended is generally shown. The syringe 10 is used in combination with a carpule 12 and a needle 14. The carpule 12 is typically formed from glass or other transparent material is of conventional design well known in the art. Carpule 12 is typically filled with a supply of fluid medication, such as novocaine, or the like. In one exemplary embodiment the outer shell 16 further comprises a window 20 to enable viewing of carpule 12, and the quantity of medicament therein.

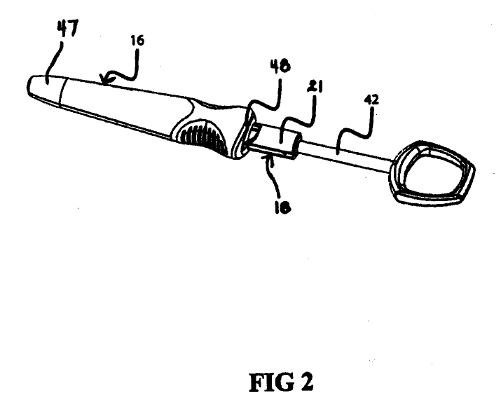

Referring to FIG. 2, an isometric view of an embodiment of a syringe 10 with a needle 14 retracted is generally shown. The syringe assembly 10 includes a hollow outer shell 16 that slidably engages at least a portion of an outer surface 21 of holder 18 and a plunger 24 slidably received in holder 18. The outer shell 18 is movable between an extended position shown in FIG. 1 and a retracted position shown in FIG. 2 for exposing the needle 14. In a further exemplary embodiment, the plunger 24 further includes a gauge for indicating the quantity of medicament in the carpule 12.

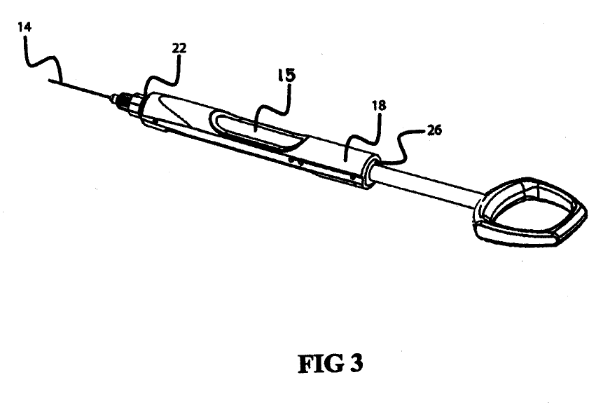

FIG. 3 illustrates an isometric view of an exemplary embodiment of a portion of the syringe 10 having the outer shell 16 removed. The holder 18 includes an aperture 15 for receiving the carpule 12 therein. A distal end 22 of the holder 18 has an aperture configured to receive a needle 14. A portion of plunger 24 extends outwardly from the proximal end 26 of the holder 18.

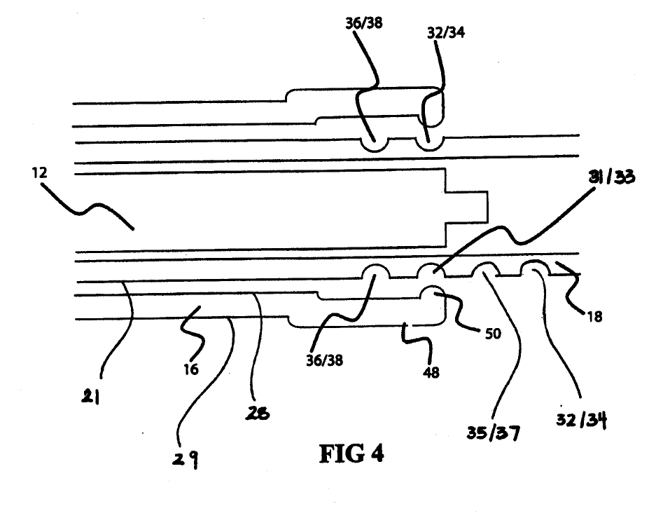

Referring to FIG. 4, a portion of the syringe assembly 10 is generally shown. The outer surface 21 of holder 18 includes at least one depression 31, 32, 35, 36 for selectively engaging a protrusion 50 located at a proximal end 48 of the outer shell 16. In one exemplary embodiment, the protrusion 50 is formed on the inner wall 28 of the outer shell 16. The outer surface 21 of holder 18 includes a first depression 36 that defines a first position 38, and a second depression 32 that defines a second position 34. The second depression 32 is proximate to the proximal end 26 of holder 18, and the first depression 36 is distal to the second depression 32. The distance between the first depression 36 and the second depression 32, and any intermediary depressions, determines the relationship of the needle 14 relative to the outer shell 16.

In another exemplary embodiment, the outer surface 21 of holder 18 further includes two intermediate depressions for selectively engaging the protrusion 50 of outer shell 16. The first intermediate depression 31 defines a first intermediate position 33 and the second intermediate depression 35 defines a second intermediate position 37. The second intermediate depression 35 is proximate to the second depression 32 and the first intermediate depression 31 is distal to each of the second intermediate depression 35 and the second depression 32. The outer shell 16 in an extended position and the second position 34 corresponds to the outer shell 16 in a retracted position.

Protrusion 50 of outer shell 18 is movable between the first 38 and second 34 positions. The first position 38 corresponds to the outer shell 16 in an extended position and the second position 34 corresponds to the outer shell 16 in a retracted position. In another exemplary embodiment, the first intermediate position 33 and second intermediate position 37 both correspond to the outer shell 16 in a partially retracted position where only a portion of needle 14 is visible. When protrusion 50 is engaged in the second intermediate depression 35, a greater portion of needle 14 is visible compared to when protrusion 50 is engaged in the first intermediate depression 31.

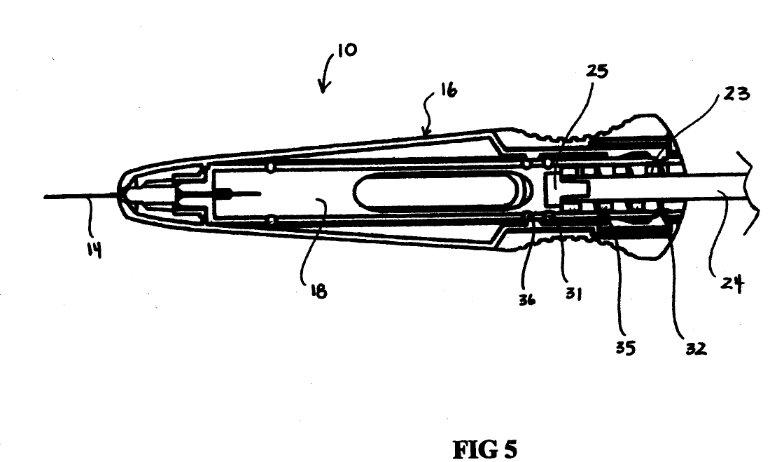

Referring to FIG. 5, a top view, partially in section, of the syringe assembly 10 is generally shown. The syringe assembly 10 further includes a spring 23 biased toward a forward end 25 of plunger 24. This forward end 25 of plunger 24 is biased forwardly by spring 23 to resiliently hold carpule 12 in holder 18. In one embodiment, the syringe assembly 10 is self-aspirating.

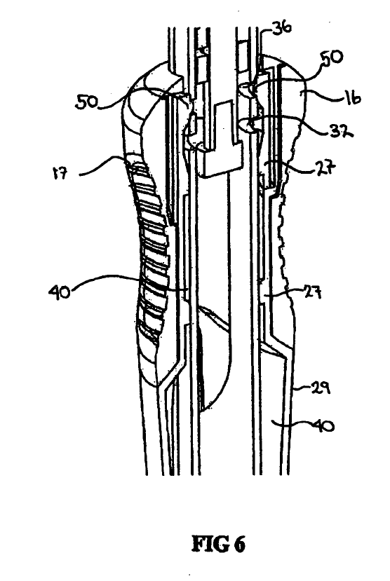

Referring to FIG. 6, a partial sectional view of the syringe assembly 10 is generally shown. The holder 18 further includes a channel 40 for continuously engaging protrusion 50 of outer shell 16. The channel 40 is formed longitudinally along at least a portion of the length of holder 18. In one embodiment, the outer shell 16 further includes at least one protuberance 27 engaging channel 40. The engagement of protuberance 27 in channel 40 prevents the rotation of holder 18 within outer shell 16. In another embodiment, the first depression 36 and second depression 32 are formed in channel 40. In a further embodiment, the outer shell further includes a grip 17 that covers a portion of the proximal end 48 of the outer wall 29 of the outer shell 16 for ease of holding the syringe assembly 10 by a user.

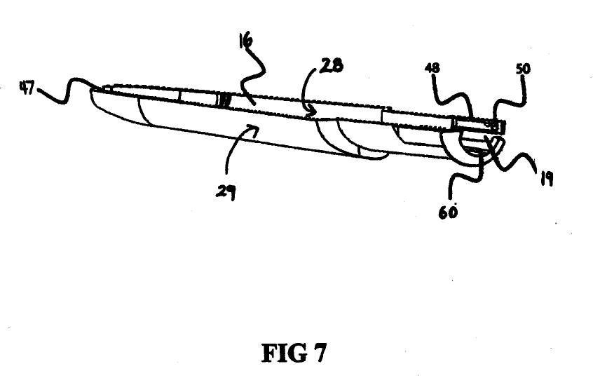

Referring to FIG. 7, an isometric view of a portion of an embodiment of the syringe assembly showing a section of an inner wall of the outer shell is generally shown. A gap 19 is provided at the proximal end 48 of outer shell 16 to allow for expansion of the outer shell 16. The outer shell further comprising an opening 47 for receiving a needle 14. In a further embodiment, the inner surface 28 of outer shell 16 including a conduit 60 extending from the proximal end 48 to the carpule 12 for allowing self-aspiration of the syringe assembly 10.

In one embodiment, a portion of a distal end 47 of outer shell 16 is textured to obscure viewing of needle 14. In another embodiment, a portion of the proximal end 48 of outer shell 16 is coated or otherwise covered with a polymeric material that is similar to that material found on toothbrushes.

In another embodiment, the syringe assembly of the present invention can be manufactured by methods known to those skilled in these arts. More particularly, the outer shell 16 can be injection molded in two identical pieces that are then joined together or in a single mold. In a further embodiment, each of holder 18 and plunger 24 may also be injection molded.

In one embodiment, the outer shell 16 is constructed from suitable flexible materials such as plastics, metals, or other materials known in the art that. The proximal end 48 of the outer shell 16 may be temporarily deformed to enable the proximal end 48 to be biased toward the holder 18.

The syringe assembly 10 to load the syringe assembly 10 with a carpule, a user pulls plunger 24 away from the proximal end 48 of outer shell 16 such that the plunger 24 is fully extended, spring 23 is in tension, and aperture 15 of holder 18 is exposed for loading a carpule 12 in holder 18. Once the carpule is loaded, the plunger 24 slides forward toward the proximal end 48 of outer shell 16, spring 23 is biased toward the carpule holding the carpule resiliently in place in holder 18, and the protrusion 50 is engaged in the first depression 36. In another embodiment, the syringe assembly 10 may be loaded with a carpule using methods known to persons skilled in the art.

In use, the syringe assembly once loaded with a carpule 12, the outer shell 16 is in an extended position such that needle 14 is in a retracted position and is completed covered by the outer shell 16 as shown in FIG. 2, and the protrusion 50 of outer shell 16 is engaged in the first depression 36 of holder 18. This is the first position 38.

The user then applies pressure to the plunger 24. The plunger 24 and holder 18 move as a unit relative to outer shell 16, and the protrusion 50 springs out of the first depression 36, and the holder 18 and plunger 24 together slidably engage the outer shell 16 sliding toward the distal end of outer shell 16 until protrusion 50 engages a first intermediate depression 31. In this first intermediate position 33 a portion of needle 14 is visible and the outer shell 16 is partially retracted. Pressure continues to be applied by the user and protrusion 50 springs out of the first intermediate depression 31, the holder 18 and plunger 24 continue to slide as a unit in the outer shell 16 until protrusion 50 engages a second intermediate depression 35. In this second intermediate position 37, a greater portion of needle 14 is visible and a greater portion of the outer shell 16 is partially retracted. Pressure continues to be applied by the user and protrusion 50 springs out of the second intermediate depression 35, the holder 18 and plunger 24 together continue to slide as a unit in outer shell 16 until protrusion 50 then engages the second depression 32. In this second position 34, the outer shell 16 is in a retracted position such that needle 14 is in an extended position as shown in FIG. 1.

Upon reaching the second depression, the user applies further pressure to plunger 24. The plunger 24 slides in holder 18 and the forward end 25 of the plunger 24 selectively engages the container 12. The continued pressure by the uses moves the plunger 24 to a dispensing position and urges the contents of the carpule 12 from the carpule 12, through the needle 14.

The above-described embodiments have been provided as examples, for clarity in understanding the invention. A person of skill in the art will recognize that alterations, modifications and variations may be effected to the embodiments described above while remaining within the scope of the invention as defined by the claims appended hereto.

1. A syringe assembly comprising:
a holder for receiving a container having a medicament;
a plunger slidably received in said holder, a forward end of said plunger for selectively engaging said container to dispense said medicament through a needle;
an outer shell having an inner surface, a portion of said inner surface slidably engaging an outer surface of said holder, said outer shell being movable between an extended position and a retracted position for exposing said needle;
wherein said plunger and said holder are movable as a unit relative to said outer shell when said outer shell moves between said extended position and said retracted position, and when said outer shell is in said retracted position, said plunger is movable to a dispensing position.

2. A syringe assembly as claimed in claim 1, a proximal end of said outer shell is biased toward said holder.

3. A syringe assembly as claimed in claim 2, wherein said proximal end includes a protrusion for selectively engaging a depression in said outer surface of said holder.

4. A syringe assembly as claimed in claim 3, wherein said protrusion of said outer shell is biased toward said depression.

5. A syringe assembly as claimed in claim 3, wherein said protrusion is formed on an inner wall of said outer shell.

6. A syringe assembly as claimed in claim 3, wherein said outer surface of said holder having a first depression and a second depression, said protrusion engaging said first depression corresponding to said extended position of said outer shell, and said protrusion engaging said second depression corresponding to said retracted position of said outer shell.

7. A syringe assembly as claimed in claim 3, wherein a channel in said outer surface of said holder continuously engages said protrusion.

8. A syringe assembly as claimed in claim 7, wherein said depression is formed in said channel.

9. A syringe assembly as claimed in claim 6, wherein said outer surface of said holder has at least one intermediate depression.

10. A syringe assembly as claimed in claim 9, wherein said protrusion engaging said intermediate depression corresponds to a partially retracted position of said outer shell.

11. A syringe assembly as claimed in claim 6, wherein said outer surface of said holder has two intermediate depressions.

12. A syringe assembly as claimed in claim 11, wherein said protrusion engaging at least one of said intermediate depressions corresponds to a partially retracted position of said outer shell.

13. A syringe assembly as claimed in claim 6, wherein said outer surface of said holder has a plurality of intermediate depressions.

14. A syringe assembly as claimed in claim 1, wherein an outer wall of said outer shell has a window formed therein.

15. A syringe assembly as claimed in claim 1, wherein said outer shell is transparent.

16. A syringe assembly as claimed in claim 1, wherein a portion of a distal end of said outer shell is textured, said textured portion configured to conceal the needle.

17. A syringe assembly as claimed in claim 1, further including a spring biased toward said container for resiliently retaining said container in said holder.

18. A syringe assembly as claimed in claim 1, wherein said syringe is self-aspirating.

19. An outer shell for a syringe comprising:
an inner surface, a portion of said inner surface slidably engaging an outer surface of a holder, said holder for receiving a container containing therein a medicament;
a proximal end, biased toward said holder for selectively engaging said outer surface of said holder;
said outer shell being movable between an extended position and a retracted position for exposing a needle;
wherein when said outer shell moves between said extended position and said retracted position, a plunger slidably received in said holder, and said plunger and said holder are movable as a unit relative to said outer shell, and when said outer shell is in said retracted position, a forward end of said plunger is movable relative to said holder in a dispensing direction to dispense said medicament through said needle.

20. An outer shell as claimed in claim 19, wherein said proximal end includes a protrusion for selectively engaging a depression in said outer surface of said holder.

21. An outer shell as claimed in claim 20, wherein said protrusion of said outer shell is biased toward said depression.

22. An outer shell as claimed in claim 20, wherein said protrusion is formed on an inner wall of said outer shell.

23. An outer shell as claimed in claim 20, wherein said outer surface of said holder has a first depression and a second depression, said protrusion engaging said first depression corresponds to said extended position of said outer shell, and said protrusion engaging said second depression corresponds to said retracted position of said outer shell.

24. An outer shell as claimed in claim 20, wherein a channel in said outer surface of said holder continuously engages said protrusion.

25. An outer shell as claimed in claim 24, wherein said depression is formed in said channel.

26. An outer shell as claimed in claim 23, wherein said outer surface of said holder has at least one intermediate depression.

27. An outer shell as claimed in claim 26, wherein said protrusion engaging said intermediate depression corresponds to a partially retracted position of said outer shell.

28. An outer shell as claimed in claim 23, wherein said outer surface of said holder has two intermediate depressions.

29. An outer shell as claimed in claim 28, wherein said protrusion engaging at least one of said intermediate depressions corresponds to a partially retracted position of said outer shell.

30. An outer shell as claimed in claim 23, wherein said outer surface of said holder has a plurality of intermediate depressions.

31. An outer shell as claimed in claim 19, wherein an outer wall of said outer shell has a window formed therein.

32. An outer shell as claimed in claim 1, wherein said outer shell is transparent.

33. An outer shell as claimed in claim 19, wherein a portion of a distal end of said outer shell is textured, to conceal the needle.

34. An outer shell as claimed in claim 19, further including a spring biased toward said container.

35. An outer shell as claimed in claim 19, wherein said syringe is self-aspirating.

36. A syringe assembly as claimed in claim 1, wherein said outer shell is constructed of a suitable flexible material that is temporarily deformable.

37. A syringe assembly as claimed in claim 36, wherein said suitable flexible material is selected from the group consisting of plastics, metals, polymeric materials compliant with a regulatory requirement, and combinations thereof.

38. A syringe assembly as claimed in claim 37, wherein the compliant polymeric material is shaped into a child-friendly shape.

39. A syringe assembly as claimed in claim 2, wherein a portion of the proximal end of said outer shell has an outer coating of a compliant polymeric material.

40. A syringe assembly as claimed in claim 1, wherein said outer shell is injection molded.

41. A syringe assembly as claimed in claim 1, wherein said syringe assembly is injection molded.

42. An outer shell as claimed in claim 19, wherein said outer shell is injection molded.
